(19) United States
(12) Patent Application Publication
(10) Pub. No.: US 2025/0052743 A1
(43) Pub. Date: Feb. 13, 2025

(54) CAPILLARY ELECTROPHORESIS OF ENCAPSULATED RNA

(71) Applicant: DH Technologies Development Pte. Ltd., Singapore (SG)

(72) Inventor: Mervin GUTIERREZ, West Covina, CA (US)

(21) Appl. No.: 18/721,329

(22) PCT Filed: Dec. 22, 2022
(86) PCT No.: PCT/IB2022/062699

§ 371 Date: Jun. 18, 2024

Related U.S. Application Data
(60) Provisional application No. 63/295,029, filed on Dec. 30, 2021.

(51) Int. Cl. G01N 33/53; C12Q 1/6806

(52) U.S. Cl. CPC G01N 33/5308; C12Q 1/6806

(57) ABSTRACT

The presently claimed and described technology provides methods for analyzing an encapsulated biomolecule by loading the encapsulated biomolecule on a capillary electrophoresis (CE) capillary, wherein the CE capillary is filled with a buffer comprising a polymer matrix; applying a voltage to the CE capillary to release the biomolecule from the encapsulating material; and detecting the biomolecule released from the encapsulating material. Kits for analyzing an encapsulated biomolecule are also disclosed.

### RELATED APPLICATIONS

The present patent application claims the priority benefit of U.S. Provisional Patent Application Ser. No. 63/295,029, filed Dec. 30, 2021, the content of which is hereby incorporated by reference in its entirety into this disclosure.

### BACKGROUND

Existing mobility-based methods of encapsulated biomolecule analysis, such as HPLC, require a separate step to remove the biomolecule from the encapsulating material. During this extraction step to release a biomolecule from an encapsulating material, such as RNA extracted from a lipid nanoparticle, variations such as manual operations, incubation duration and temperature, mixing conditions, and reagent source can result in suboptimal recovery or quality of extracted biomolecules.

Methods allowing for direct release and analysis of encapsulated biomolecules would therefore be advantageous.

### SUMMARY

In a first aspect, the disclosure provides a method for analyzing an encapsulated biomolecule, the method comprising: loading the encapsulated biomolecule on a capillary electrophoresis (CE) capillary, wherein the CE capillary is filled with a buffer comprising a polymer matrix; applying a voltage to the CE capillary to release the biomolecule from the encapsulating material; and detecting the biomolecule released from the encapsulating material.

In some aspects of the method, the polymer matrix comprises a chaotrope.

In some aspects of the method, detecting the biomolecule released from the encapsulating material produces a set of corresponding values, and the method further comprises quantifying the biomolecule released from the encapsulating material using the corresponding values.

In some aspects of the method, the method further comprises adding a fluorescent dye to the polymer matrix and/or to a buffer disposed within the CE capillary, wherein the fluorescent dye binds the biomolecule resulting in a fluorescently labeled biomolecule.

In some aspects of the method, the fluorescent dye is a cyanine-based dye.

In some aspects of the method, the method further comprises heating the encapsulated biomolecule prior to loading the encapsulated biomolecule on the CE capillary.

In some aspects of the method, the encapsulated biomolecule is heated at a temperature between about 40° C. to about 90° C., alternatively at a temperature between about 45° C. to about 85° C., alternatively at a temperature between about 50° C. to about 80° C., alternatively at a temperature between about 55° C. to about 78° C., alternatively at a temperature between about 60° C. to about 77° C., alternatively at a temperature between about 65° C. to about 75° C., alternatively at a temperature between about 68° C. to about 74° C., alternatively at a temperature between about 69° C. to about 73° C., alternatively at a temperature of about 70° C.

In some aspects of the method, the encapsulated biomolecule is heated for at least 2 minutes, alternatively at least 3 minutes, alternatively at least 4 minutes, alternatively at least 5 minutes.

In some aspects of the method, the method further comprises cooling the encapsulated biomolecule after heating.

In some aspects of the method, the method further comprises treating the encapsulated biomolecule with a denaturing agent prior to loading the encapsulated biomolecule on the CE capillary.

In some aspects of the method, the method further comprises detecting the encapsulating material.

In some aspects of the method, the biomolecule is a polynucleotide.

In some aspects of the method, the polynucleotide is an mRNA encoding a polypeptide.

In some aspects of the method, the encapsulating material is a lipid nanoparticle comprising one or more of an ionizable cationic lipid, a PEGylated lipid, a phospholipid, and/or cholesterol.

In some aspects of the method, the biomolecule is a protein or a polynucleotide.

In some aspects of the method, the encapsulating material is a viral vector.

In some aspects of the method, the polymer matrix is a cross-linked polymer, a linear polymer, a branched polymer, linear polyacrylamide, polyethylene oxide, polyethylene glycol, dextran, or pullulan.

In some aspects of the method, detecting the biomolecule utilizes a fluorescence detector.

In some aspects of the method, the fluorescence detector is a laser-induced fluorescence (LIF) detector, a lamp-based fluorescence detector, or a native fluorescence detector.

In some aspects of the method, the chaotrope is n-butanol, ethanol, guanidinium chloride, lithium acetate, magnesium chloride, 2-propanol, sodium dodecyl sulfate, thiourea, or urea.

In some aspects of the method, the chaotrope is urea.

In some aspects of the method, the buffer further comprises the chaotrope.

In another aspect, the disclosure provides a kit for analyzing an encapsulated biomolecule, the kit comprising: a fluorescent dye; a CE capillary; a buffer comprising a polymer matrix; at least one internal standard; and instructions for use.

In some aspects of the kit, the polymer matrix comprises a chaotrope.

In some aspects of the kit, the buffer further comprises the chaotrope.

In some aspects of the kit, the chaotrope is n-butanol, ethanol, guanidinium chloride, lithium acetate, magnesium chloride, 2-propanol, sodium dodecyl sulfate, thiourea, or urea.

In some aspects of the kit, the chaotrope is urea.

In some aspects of the kit, the polymer matrix is a cross-linked polymer, a linear polymer, a branched polymer, linear polyacrylamide, polyethylene oxide, polyethylene glycol, dextran, or pullulan.

In some aspects of the kit, the fluorescent dye is cyanine-based dye.

In some aspects of the kit, the CE capillary is a bare fused silica capillary.

In some aspects of the kit, the CE capillary is a neutral coated capillary.

Other aspects and features of the present disclosure will become apparent to those ordinarily skilled in the art upon review of the following description of specific embodiments of the disclosure in conjunction with the accompanying figures.

### BRIEF DESCRIPTION OF THE FIGURES

Embodiments of the present disclosure will now be described, by way of example only, with reference to the attached Figures.

FIGS. 1A and 1B illustrate inlet reagent tray layout and outlet reagent tray layout for a sample separation method.

FIGS. 2A-2C illustrate instrument settings for pressure sample injection and separation.

FIG. 3 illustrates an electropherogram of an ssRNA ladder.

FIG. 4A illustrates an electropherogram of free mRNA diluted in deionized formamide.

FIG. 4B illustrates an electropherogram of LNP-encapsulated mRNA diluted in deionized formamide.

FIG. 4C illustrates an electropherogram of an empty LNP diluted in deionized formamide.

FIG. 4D illustrates an electropherogram of an ssRNA diluted in deionized formamide.

FIG. 5 illustrates an electropherogram of an ssRNA ladder with the fragment sizes labeled. The inset of FIG. 5 displays a polynomial fit for a select region of markers that include 0.5, 1, 2, and 3 kilobases in size.

FIG. 6A illustrates an electropherogram that displays the calculated size of free mRNA diluted in deionized formamide.

FIG. 6B illustrates an electropherogram that displays the calculated size of LNP-encapsulated mRNA treated with a detergent and diluted in deionized formamide.

FIG. 6C illustrates an electropherogram that displays the calculated size of empty LNP treated with a detergent and diluted in deionized formamide.

FIG. 7 illustrates an electropherogram comparing detergent-treated LNP-encapsulated mRNA with non-detergent treated LNP-encapsulated mRNA diluted in deionized formamide.

FIG. 8 illustrates an electropherogram of detergent-treated LNP-encapsulated mRNA and non-detergent treated LNP-encapsulated mRNA diluted in nuclease-free water or deionized formamide.

FIGS. 9A-9B illustrate an electropherogram of LNP-encapsulated mRNA diluted in nuclease-free water without heat treatment (FIG. 9A) or with denaturing at 70° C. (FIG. 9B).

FIG. 10 illustrates an electropherogram of heat-denatured and non-heat denatured LNP-encapsulated mRNA and empty LNP.

FIG. 11 illustrates an electropherogram of heat-denatured LNP-encapsulated mRNA and empty LNP diluted in nuclease-free water.

### DETAILED DESCRIPTION

It is to be understood that this disclosure is not limited to the particular methodology, protocols, and reagents described herein and as such may vary. It is also to be understood that the terminology used herein is for the purpose of describing particular embodiments only and is not intended to limit the scope of the present disclosure or the appended claims.

As used herein and in the appended claims, the singular forms “a,” “an,” and “the” include plural reference unless the context clearly indicates otherwise.

The term “about” is used in connection with a numerical value throughout the specification and the claims denote an interval of accuracy, familiar and acceptable to a person skilled in the art. In general, such an interval of accuracy is +/−10%.

Unless defined otherwise, all technical and scientific terms used herein have the same meaning as commonly understood to one of ordinary skill in the art to which this disclosure belongs.

The disclosure generally relates to methods and kits for measuring mobility (and therefore fragment size) of encapsulated biomolecules without the need for a separate step to release the biomolecule from the encapsulating material.

In a first aspect, the disclosure provides methods of analyzing encapsulated biomolecules. As used herein, “encapsulation” refers to the process of stabilizing a biomolecule by depositing (e.g., coating) the biomolecule in a carrier material. Encapsulation preserves the biological, physical, and/or chemical properties of the biomolecule, and facilitates its release or delivery under established or desired conditions. An “encapsulated biomolecule” is a biomolecule having undergone the process of encapsulation. “Carrier material”, “shell”, “shell material”, “wall material”, “coating material”, “encapsulating material”, “delivery material”, “delivery vehicle”, and “encapsulating agent” can be used interchangeably, and refer to the material in which a biomolecule is encapsulated. In the context of encapsulation, “biomolecule”, “active molecule”, “active agent”, and “active material” can be used interchangeably, and refer to the material being encapsulated.

Biomolecules of the present disclosure include polynucleotides. “Polynucleotides”, “nucleic acid sequence”, “nucleotide sequence”, and “polynucleotide” can be used interchangeably, and refer to a continuous sequence of nucleic acids. Non-limiting examples of nucleic acids include deoxyribonucleic acid (DNA), ribonucleic acid (RNA), single-stranded DNA (ssDNA), double-stranded DNA (dsDNA), microRNA (miRNA), and messenger RNA (mRNA).

Biomolecules of the present disclosure also include polypeptides. “Polypeptide”, “protein”, and “peptide” can be used interchangeably, and refer to polymers of amino acids of any length.

In some aspects, the encapsulating material is a nanoparticle formulation comprising, but not limited to, poly(lactic-co-glycolic acid) (PLGA) microspheres, lipidoids, lipoplex, liposome, polymers, carbohydrates (including simple sugars), cationic lipids, and combinations thereof.

In exemplary aspects, the encapsulating material is a lipid nanoparticle (LNP). Lipid nanoparticle formulations are known to one of ordinary skill, and include formulations comprising an ionizable cationic lipid, a PEGylated lipid, a phospholipid, and/or cholesterol.

In some aspects, the encapsulating material is a viral vector such as, but not limited to, a lentivirus, an adenovirus, an adeno-associated virus (AAV), a herpes simplex virus, or a retrovirus.

In exemplary aspects, the biomolecule is mRNA encapsulated in a lipid nanoparticle.

In some aspects, a method for analyzing encapsulated biomolecules comprises: loading an encapsulated biomolecule on a capillary electrophoresis (CE) capillary, the CE capillary being filled with a buffer comprising a polymer matrix; applying a voltage to the CE capillary to release the biomolecule from the encapsulating material; and detecting the biomolecule released from the encapsulating material.

In some aspects, a method for analyzing encapsulated biomolecules comprises: loading an encapsulated biomolecule on a capillary electrophoresis (CE) capillary, the CE capillary being filled with a buffer comprising a polymer matrix, wherein a fluorescent dye is added to the polymer matrix and/or to the buffer to bind the biomolecule, which results in a fluorescently labeled biomolecule; applying a voltage to the CE capillary to release the biomolecule from the encapsulating material; and detecting the biomolecule released from the encapsulating material.

In some aspects, a method for analyzing encapsulated biomolecules comprises: optionally heating an encapsulated biomolecule; optionally cooling the heated encapsulated biomolecule; loading the encapsulated biomolecule on a capillary electrophoresis (CE) capillary, the CE capillary being filled with a buffer comprising a polymer matrix; applying a voltage to the CE capillary to release the biomolecule from the encapsulating material; and detecting the biomolecule released from the encapsulating material.

In some aspects, a method for analyzing encapsulated biomolecules comprises: optionally heating an encapsulated biomolecule; optionally cooling the heated encapsulated biomolecule; loading the encapsulated biomolecule on a capillary electrophoresis (CE) capillary, the CE capillary being filled with a buffer comprising a polymer matrix, wherein a fluorescent dye is added to the polymer matrix and/or to the buffer to bind the biomolecule, which results in a fluorescently labeled biomolecule; applying a voltage to the CE capillary to release the biomolecule from the encapsulating material; and detecting the biomolecule released from the encapsulating material.

In some aspects, a method for analyzing encapsulated biomolecules comprises: optionally denaturing an encapsulated biomolecule with heat, a denaturing agent (i.e., a non-heat denaturant), or both heat and a denaturing agent; optionally cooling, if heat is used as a denaturant, the heated encapsulated biomolecule; loading the encapsulated biomolecule on a capillary electrophoresis (CE) capillary, the CE capillary being filled with a buffer comprising a polymer matrix; applying a voltage to the CE capillary to release the biomolecule from the encapsulating material; and detecting the biomolecule released from the encapsulating material.

In some aspects, a method for analyzing encapsulated biomolecules comprises: optionally denaturing an encapsulated biomolecule with heat, a denaturing agent (i.e., a non-heat denaturant), or both heat and a denaturing agent; optionally cooling, if heat is used as a denaturant, the heated encapsulated biomolecule; loading the encapsulated biomolecule on a capillary electrophoresis (CE) capillary, the CE capillary being filled with a buffer comprising a polymer matrix, wherein a fluorescent dye is added to the polymer matrix and/or to the buffer to bind the biomolecule, which results in a fluorescently labeled biomolecule; applying a voltage to the CE capillary to release the biomolecule from the encapsulating material; and detecting the biomolecule released from the encapsulating material.

In any of the above methods, the polymer matrix, the buffer, or both the polymer matrix and the buffer can include a chaotrope. “Chaotrope” or “chaotropic agent” as used herein refer to an agent that disrupts hydrogen bonding between water molecules, and in the context of the disclosure, disrupts the hydration shell and hydrophobic interactions of macromolecules (e.g., nucleic acids and polypeptides) in an aqueous solution, thus weakening the structure of the macromolecule. Suitable chaotropes of the method include, n-butanol, ethanol, guanidinium chloride, lithium acetate, magnesium chloride, 2-propanol, sodium dodecyl sulfate, thiourea, and urea.

In exemplary aspects, the chaotrope is urea.

In some aspects, the chaotrope in the buffer and/or the polymer matrix is about 4 to about 8 M, alternatively about 5 to about 7 M, alternatively about 6 to about 7 M, alternatively about 7 M.

In some aspects, when the biomolecule is mRNA encapsulated in a lipid nanoparticle, the chaotropic agent disrupts hydrogen bonding to reduce the stability of the lipid nanoparticle network that shields the mRNA.

In some aspects, when a fluorescent dye is added to the polymer matrix, the buffer, or both the polymer matrix and the buffer, the fluorescent dye is a cyanine-based dye. Cyanine-based dyes of the disclosure include, not are not limited to, Cy2, Cy3, Cy3B, Cy3.5, Cy5, Cy5,5, Cy7, SYBR Green I, SYBR Green II, PicoGreen, Thiazole orange, and Oxazole yellow.

In exemplary aspects, the fluorescently labeled dye is SYBR Green I or SYBR Green II.

In some aspects, when the method includes heating an encapsulated biomolecule, the encapsulated biomolecule can be heated at a temperature between about 40° C. to about 90° C., alternatively at a temperature between about 45° C. to about 85° C., alternatively at a temperature between about 50° C. to about 80° C., alternatively at a temperature between about 55° C. to about 78° C., alternatively at a temperature between about 60° C. to about 77° C., alternatively at a temperature between about 65° C. to about 75° C., alternatively at a temperature between about 68° C. to about 74° C., alternatively at a temperature between about 69° C. to about 73° C., alternatively at a temperature of about 70° C.

In some aspects, when the method includes heating an encapsulated biomolecule, the encapsulated biomolecule can be heated for at least 2 minutes, alternatively at least 3 minutes, alternatively at least 4 minutes, alternatively at least 5 minutes.

In some aspects, when the method includes denaturing an encapsulated biomolecule with a denaturing agent (i.e., a non-heat denaturant), the denaturing agent can comprise acetic acid, trichloroacetic acid, hydrochloric acid, sulfosalicylic acid, nitric acid, sodium bicarbonate, ethanol, formaldehyde, glutaraldehyde, urea, guanidinium chloride, lithium perchlorate, sodium dodecyl sulfate (SDS), dimethyl sulfoxide (DMSO), 2-mercaptoethanol, Dithiothreitol (DTT), tris(2-carboxyethyl) phosphine) (TCEP), formamide, guanidine, sodium salicylate, propylene glycol, and urea.

In some aspects, the denaturing agent is a nonionic detergent such as Triton X-100 or Tergitol 15-S-20.

In some aspects, non-limiting examples of the polymer matrix include cross-linked polymer, linear polymers, branched polymers, linear polyacrylamide, polyethylene oxide, polyethylene glycol, dextran, polyvinylpyrrolidone, and pullulan.

In the methods described herein, when a voltage is applied to the CE capillary, the encapsulated biomolecule is mobilized based on overall charge and migrates towards a detector. During migration, the biomolecule progressively releases from the encapsulation, such that the biomolecule is physically separated from the encapsulation.

In some aspects, the field strength during mobilization of encapsulated biomolecules is about 200 to about 1000 V/cm, alternatively about 200 to about 750 V/cm, alternatively about 200 to about 500 V/cm, alternatively about 200 to about 250 V/cm, alternatively about 200 V/cm.

In some aspects, the biomolecule is LNP-encapsulated mRNA. As the LNP-encapsulated mRNA move into the chaotropic gel buffer through an electric field, the encapsulation is destabilized, allowing for mRNA release. The negatively charged mRNA then moves toward the positive anode and is visualized by a detector.

Encapsulated biomolecules can be analyzed using capillary zone electrophoresis, capillary gel electrophoresis, capillary isoelectric focusing, micellar electrokinetic capillary chromatography, or capillary electrochromatography. In some aspects, the method uses capillary gel electrophoresis (CGE), which separates and releases encapsulated biomolecules by size and detects the released biomolecules using a fluorescent dye that binds to the biomolecules. In some aspect, the method uses capillary zone electrophoresis (CZE), which separates and releases encapsulated biomolecules by electrophoretic mobility, which is directly proportional to the charge on the biomolecule and inversely proportional to the viscosity of the solvent and radius of the atom.

As used herein, “capillary” refers to a channel, tube, or other structure capable of supporting a volume of separation medium for performing electrophoresis. Capillary geometry can vary and includes structures having circular, rectangular, or square cross-sections, channels, groves, plates, etc. that can be fabricated by technologies known in the art. Capillaries of the present disclosure can be made of materials such as, but not limited to, silica, fused silica, quartz, silicate-based glass such as borosilicate glass, phosphate glass, or alumina-containing glass, and other silica-like materials. In some aspects, the methods can be adapted and used in any generally known electrophoresis platform such as, for example, electrophoresis devices comprising single or multiple microfluidic channels, etched microfluidic capillaries, as well as slab gel and thin-plate gel electrophoresis.

In some aspects, the capillary is an uncoated capillary.

In some aspects, the capillary is a coated capillary. For example, a capillary can be coated to shield or minimize electrostatic interactions. Shielding can comprise non-permanent, replaceable polymeric hydrophilic coatings that adsorb to the capillary surface or permanent hydrophilic coatings comprising linear polyacrylamide or polyvinylalcohol that covalently bind the capillary surface.

The detector can be a UV detector or a fluorescence detector, such as a laser-induced fluorescence (LIF) detector, a lamp-based fluorescence detector, or a native fluorescence detector. The desired quantitation sensitivity will determine the type of detector used. LIF detection offers the benefit of about a 100-fold increase in sensitivity, yet it also requires additional sample manipulation.

In some aspects, the method is used in an amplification-free workflow, a high-throughput screening application, or a rapid screening workflow. The method can also be used to analyze at least two encapsulated biomolecules simultaneously, alternatively at least three encapsulated biomolecules, alternatively at least four encapsulated biomolecules, alternatively at least five encapsulated biomolecules, alternatively at least six encapsulated biomolecules, alternatively at least seven encapsulated biomolecules, alternatively at least eight encapsulated biomolecules.

Once released or separated from the encapsulation, the biomolecule can undergo further analysis. In some aspects, the released encapsulation, that is, the encapsulating material can be detected and analyzed using a dye or labeling moiety that binds the encapsulating material.

In some aspects, detecting the released biomolecules, the released encapsulating material, or both produces a set of corresponding values that can be used to quantify or otherwise analyze the released biomolecules and/or released encapsulating material. In some aspects, these corresponding values can be plotted on an electropherogram.

An “electropherogram” refers to a series of peaks that can be converted to determine size and/or quantity of a sample. Peaks are integrated for area as a measure of quantity, and can be corrected for mobility differences between different sized peaks. In some aspects, when the biomolecules are nucleic acids, a nucleic acid ladder comprising nucleic acid fragments of known size can be run before, during, or after sample(s) of interest.

In another aspect, the disclosure provides a kit for analyzing encapsulated biomolecules according to the methods described herein. In some aspects, a kit for analyzing encapsulated biomolecules comprises: a fluorescent dye; a CE capillary; a buffer comprising a polymer matrix; at least one internal standard; and instructions for use.

In some aspects, the polymer matrix, the buffer, or the polymer matrix and the buffer comprise a chaotrope.

In some aspects, the CE capillary of the kit is a bare fused silica (BFS) capillary.

In some aspects, the CE capillary of the kit is a neutral coated capillary.

While the present disclosure has been described with reference to certain embodiments, it will be understood by those skilled in the art that various changes may be made and equivalents may be substituted without departing from the scope of the present disclosure or appended claims. In addition, many modifications may be made to adapt a particular situation or material to the teachings of the present disclosure without departing from its scope. Therefore, it is intended that the present disclosure not be limited to the particular embodiments disclosed, but that the present disclosure will include all aspects falling within the scope of the appended claims.

### EXAMPLES

### Example 1: Materials

Size ladder: ssRNA ladder 0.5-9 kb (New England BioLabs, PN N0362S), low-range ssRNA ladder 50-1000 bases (New England BioLabs, PN N0364S), Sample Loading Solution (Sciex, PN 608082).

Detergents: Triton X-100, Tergitol 15-S-20 (EU REACH compliant).

Sample diluent: nuclease free water (Invitrogen, PN AM9932), Sample Loading Solution (Sciex, PN 608082).

Capillary cartridge: Bare-fused silica capillary (20 cm to window; 30.2 cm total) LIF Cartridge Probe Guide (SCIEX, PN: 721126), LIF Cartridge Aperture Plug Assembly (SCIEX, PN: 721125)

SinTEF samples: Empty LNP (LNP-MC3), CleanCapFluc-mRNA (mRNA only, length 1879 bases), mRNA-LNP-1-MC3-201604 (LNP-mRNA).

Fluorescent label: SYBR Green II RNA gel stain, 10,000× concentrate in DMSO (ThermoFisher, PN: S7564).

Instrument and software: PA 800 Plus Pharmaceutical Analysis System (SCIEX) equipped with a solid-state laser and PMT detector for LIF detection. The excitation wavelength was at 488 nm and the emission wavelength was at 520 nm. Data acquisition and analysis were performed using BioPhase Analysis Software.

### Example 2: Sample Preparation

Preparation of buffer comprising a polymer matrix: Urea was added to a pre-manufactured buffer comprising a polymer matrix at a final concentration of 7 M. SYBR Green II was diluted 1:100 in DMSO (5 uL dye in 495 uL DMSO, and further diluted 1:500 in buffer (10 uL of 1:100 diluted dye in 5 mL buffer).

Preparation of RNA ladders: 2 μL of ssRNA ladder was added to 48 μL of sample loading solution. The diluted ladder was heated at 70° C. for 5 minutes in a thermal cycler and then immediately cooled in an ice bath. For separation on PA 800 Plus Pharmaceutical Analysis System, 50 μL of the RNA ladder was transferred to each well on the sample plate before the sequence run.

Preparation of LNP-encapsulated mRNA: LNP-encapsulated mRNA samples (100 ng/ml) were prepared in 0.1% Triton X-100 in TE buffer, 0.1% Tergitol 15-S-20 in TE buffer, or TE buffer without detergent. LNPs contained the cationic lipid MC3. Samples comprised 20% by volume LNP-encapsulated mRNA and 80% by volume buffer with a final detergent concentration of 0.1%. Samples were then diluted 1:10 in nuclease-free water or deionized formamide. The diluted samples were heated at 70° C. for 5 minutes in a thermal cycler and then immediately cooled in an ice bath. 50-200 μL of the sample was transferred to a sample vial and placed on the PA 800 Plus Pharmaceutical Analysis System, which was held at 10° C.

### Example 3: Reagent Preparation

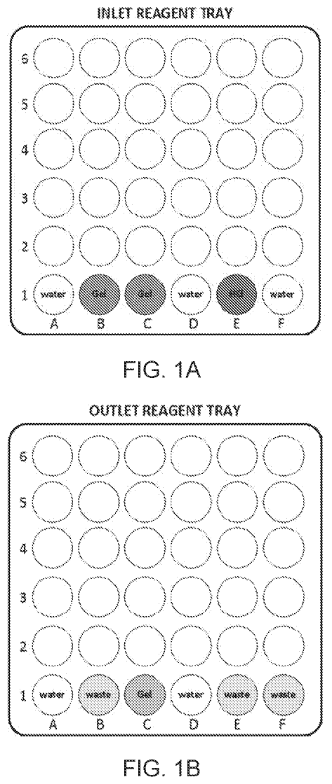

For every 10 separations, in a conical tube, 10 μL of SYBR Green II (500×) was added to 5 mL of RNA 9000 Nucleic Acid Gel and mixed by tube inversion. Reagent trays were prepared as shown in FIG. 1A (inlet tray) and FIG. 1B (outlet tray).

### Example 4: Instrument Setup & CE Separation Method

The PA 800 Plus Pharmaceutical Analysis System was prepared as follows: install LIF optics; inspect and clean manifold block, electrodes, and/or injectors; install BFS capillary cartridge with LIP aperture; perform LIF calibration to set calibration correction factor (CCF); set to 15 RFU target for 50 μm ID capillary; run conditioning method at 20° C. capillary temperature; run sample separation methods at 200 V/m and 30° C. capillary temperature; run shut down method at 20° C. capillary temperature; and store capillary at 2-8° C.

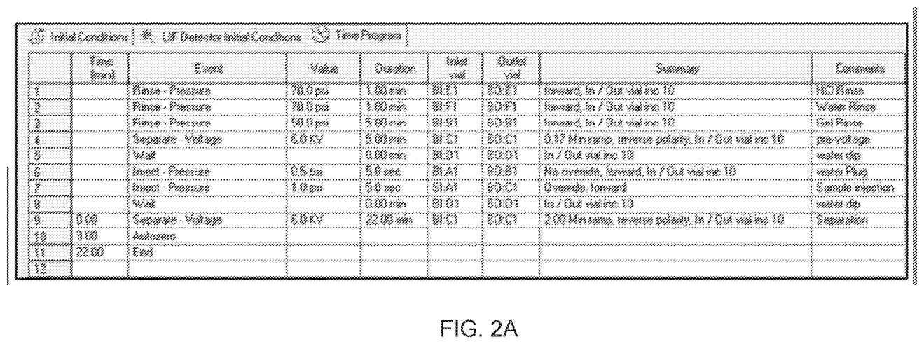

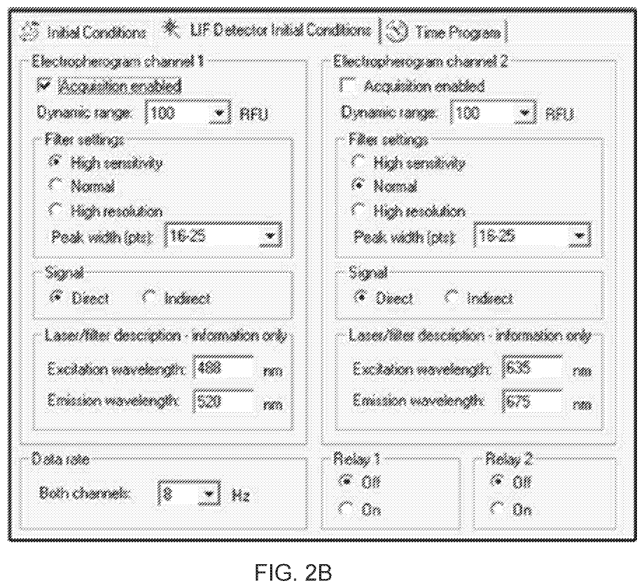

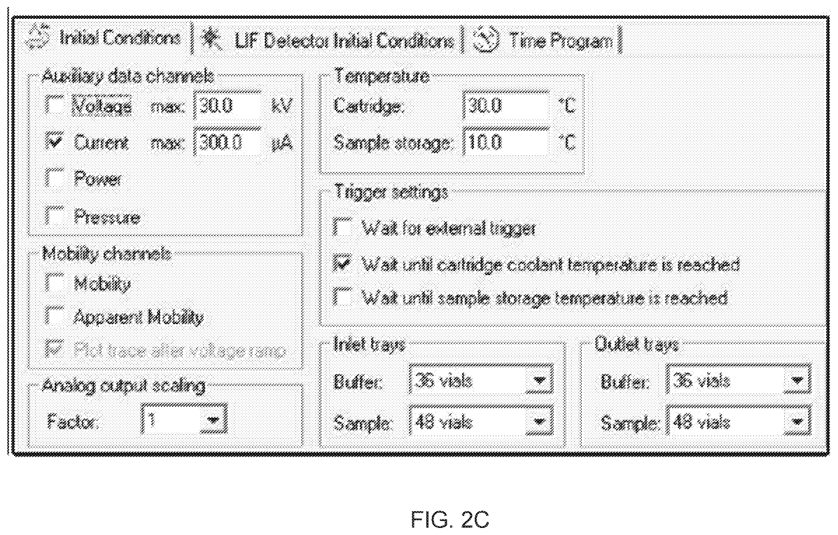

FIGS. 2A-2C illustrate instrument settings for pressure sample injection and separation.

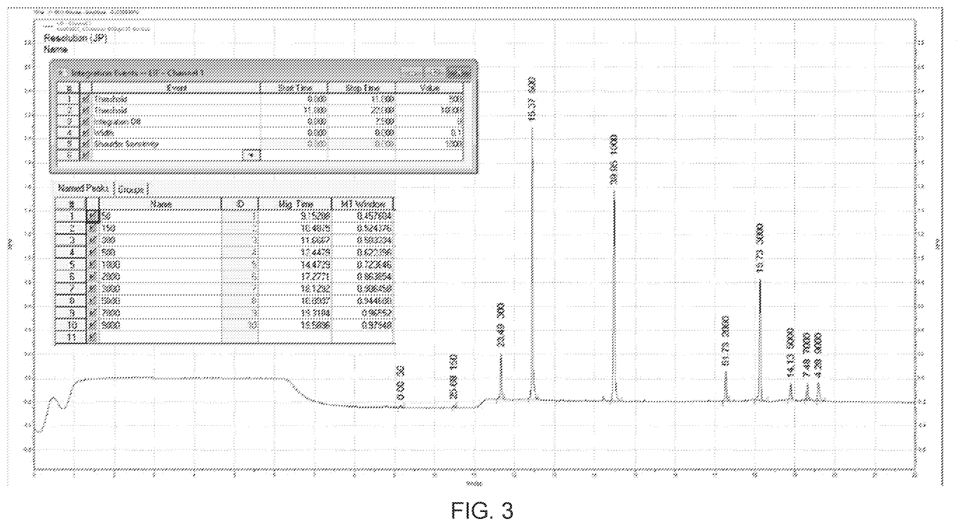

FIG. 3 illustrates an electropherogram of the ssRNA ladder with integrated peaks at 50, 150, 300, 500, 1000, 2000, 3000, 5000, 7000, and 9000 bases.

### Example 5: CE Separation of LNP-Encapsulated mRNA

To examine CE separation of detergent-treated LNP-encapsulated mRNA, CE separation according to Example 4 was performed on free mRNA (CleanCap pFLuc mRNA diluted in Sample Loading Solution), LNP-encapsulated mRNA (MC3 treated with 0.1% Triton X-100 and diluted in Sample Loading Solution), empty LNP (diluted in Sample Loading Solution), and an ssRNA ladder (diluted in Sample Loading Solution).

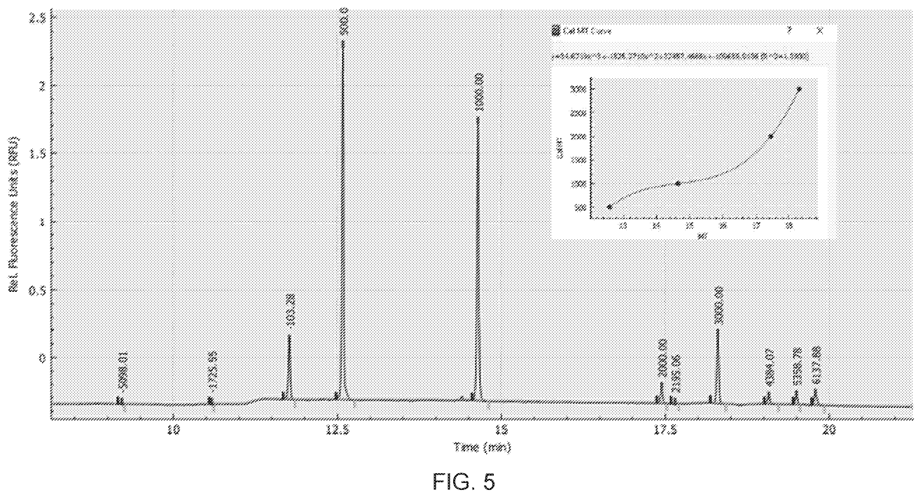

An ssRNA ladder was run prior to sample analysis, and a polynomial model was utilized for size determination of unknown samples. FIG. 5 illustrates an electropherogram of the ssRNA ladder labeled with SYBR Green II dye. The inset of FIG. 5 displays a polynomial fit for a select region of markers that include 0.5, 1, 2, and 3 kilobases in size. This polynomial model was used to calculate the size of unknown sample peaks using the migration time of runs adjacent to the standard run.

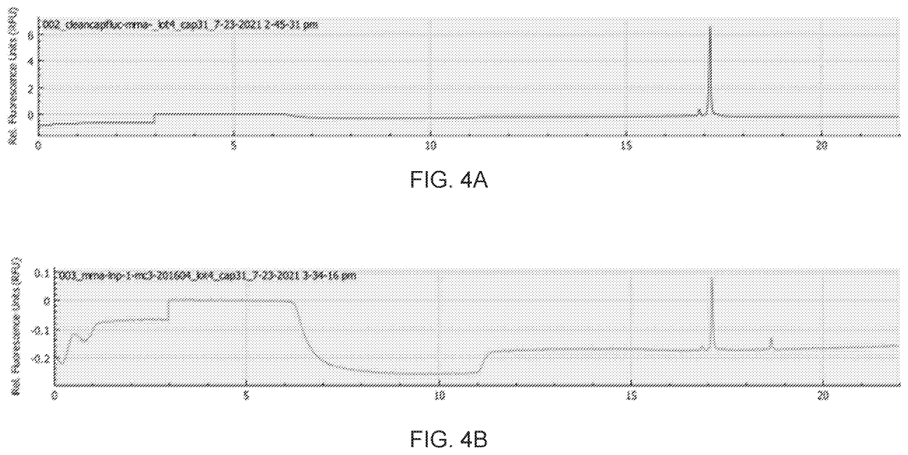

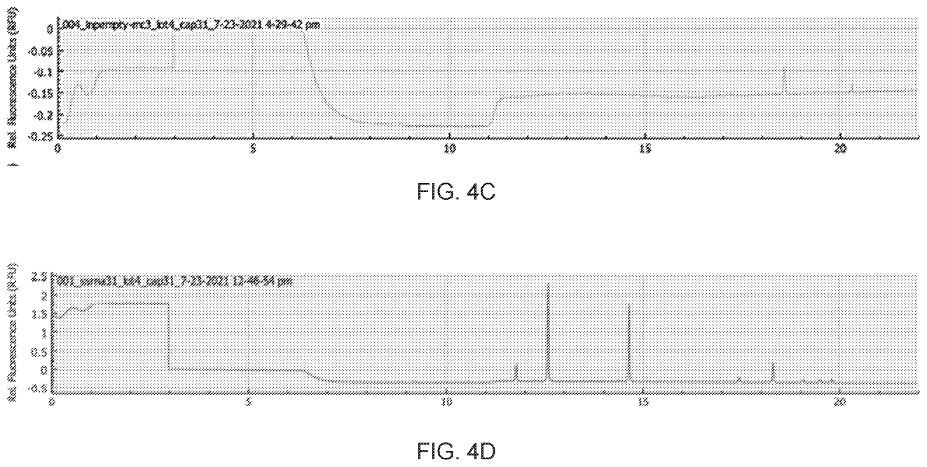

FIG. 4A is an electropherogram depicting size separation of free mRNA by CE. Free mRNA displayed a single main peak and a secondary, smaller length peak.

FIG. 4B is an electropherogram depicting size separation of LNP-encapsulated mRNA by CE. LNP-encapsulated mRNA exhibited effective release of mRNA from encapsulation as demonstrated by the resulting electropherogram that displayed a main peak and a secondary, smaller length peak, similar to the free mRNA. Another non-specific higher molecular weight peak was also detected.

FIG. 4C is an electropherogram depicting size separation of an empty LNP by CE. No peak was detected at the known size region for mRNA. A higher molecule weight peak was present, which suggests LNP-related components bind the fluorescent dye and result in a non-specific peak.

FIG. 4D is an electropherogram depicting size separation of ssRNA by CE. The size of RNA fragments on the electropherogram from left to right are 0.3 kb, 0.5 kb, 1 kb, 2 kb, 3 kb, 5 kb, 7 kb, and 9 kb.

These data demonstrate CE separation of detergent-treated LNP-encapsulated mRNA resulted in release of mRNA from the LNP, and the released mRNA exhibited a peak comparable to free mRNA.

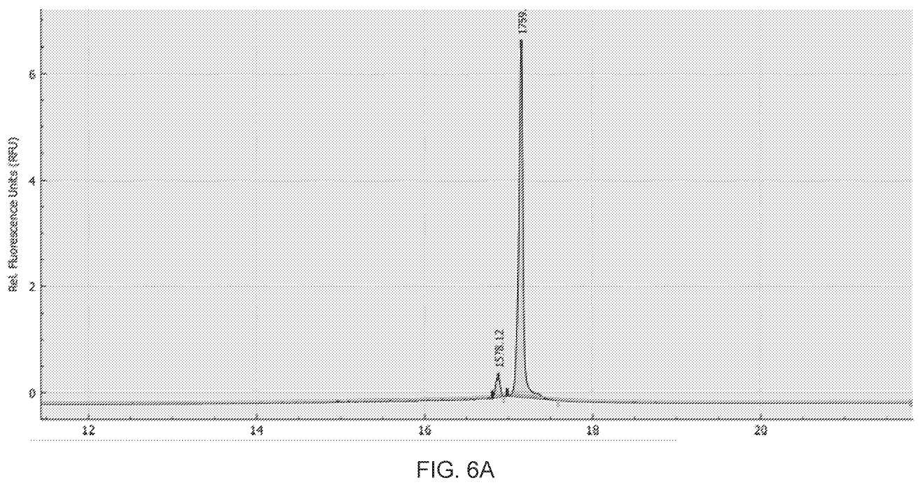

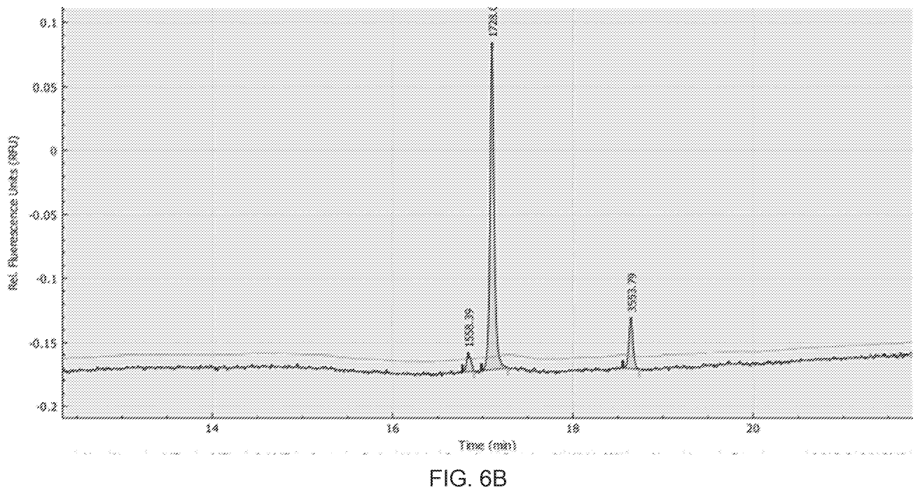

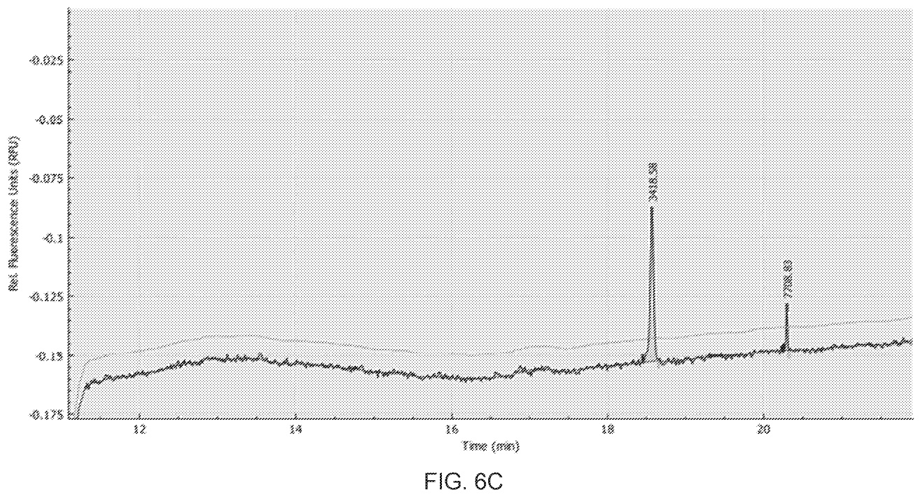

FIG. 6A is an electropherogram that displays the calculated size of free mRNA peaks based on the ssRNA ladder.

FIG. 6B is an electropherogram that displays the calculated size of LNP-encapsulated mRNA peaks based on the ssRNA ladder.

FIG. 6C is an electropherogram that displays the calculated size of empty LNP peaks based on the ssRNA ladder.

These data demonstrate polynomial modeling of the ssRNA ladder can determine sample size. Peaks can be integrated to determine quantitative data including peak area and peak height.

### Example 6: CE Separation of LNP-Encapsulated mRNA with and without Detergent

To examine the effect of detergent on CE separation of LNP-encapsulated mRNA, LNP-encapsulated mRNA was treated with 0.1% Triton X-100 or 0.1% Tergitol 15-S-20. Untreated LNP-encapsulated mRNA and empty LNP served as controls. All samples were diluted in Sample Loading Solution, and CE separation was performed according to Example 4.

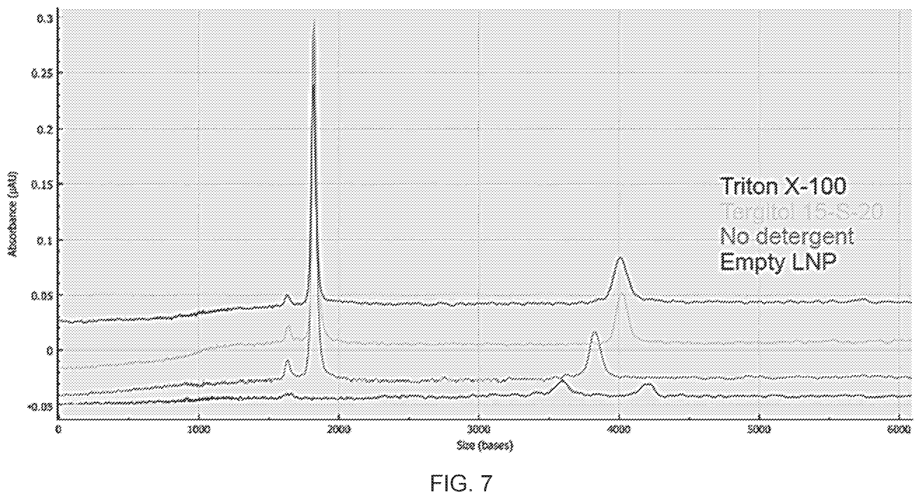

FIG. 7 is an electropherogram comparing detergent-treated (Triton X-100 or Tergitol 15-S-20) LNP-encapsulated mRNA treated with non-detergent treated LNP-encapsulated mRNA diluted with deionized formamide. The x-axis is converted from migration time to size using the applied polynomial fit from an adjacently run ssRNA ladder.

These data demonstrate the separation of detergent-treated LNP-encapsulated mRNA is comparable to non-detergent treated LNP-encapsulated mRNA.

### Example 7: CE Separation of LNP-Encapsulated mRNA Diluted in Nuclease-Free Water

LNP-encapsulated mRNA was prepared according to Example 6 and compared to non-detergent treated LNP-encapsulated mRNA diluted in nuclease-free water.

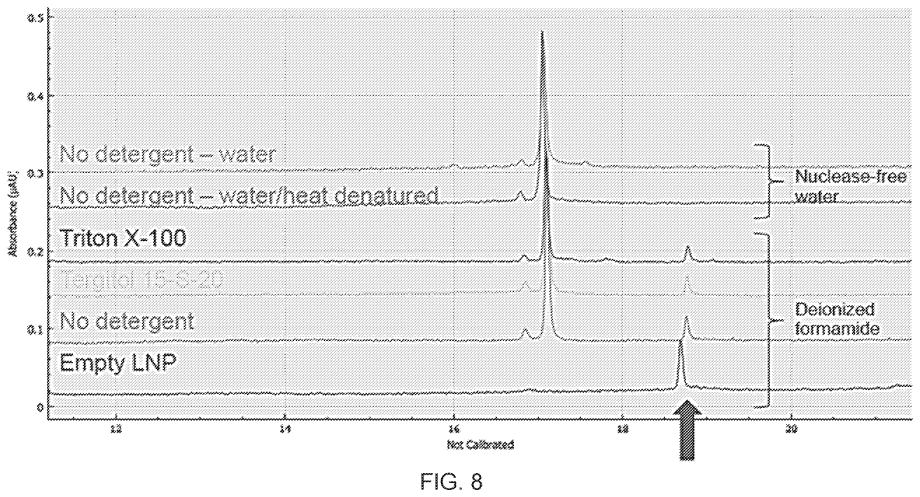

FIG. 8 illustrates an electropherogram of detergent-treated LNP-encapsulated mRNA and non-detergent treated LNP-encapsulated mRNA diluted in nuclease-free water or Sample Loading Solution, which contains deionized formamide.

These data demonstrate samples diluted in deionized formamide exhibit a non-specific peak (arrow in FIG. 8).

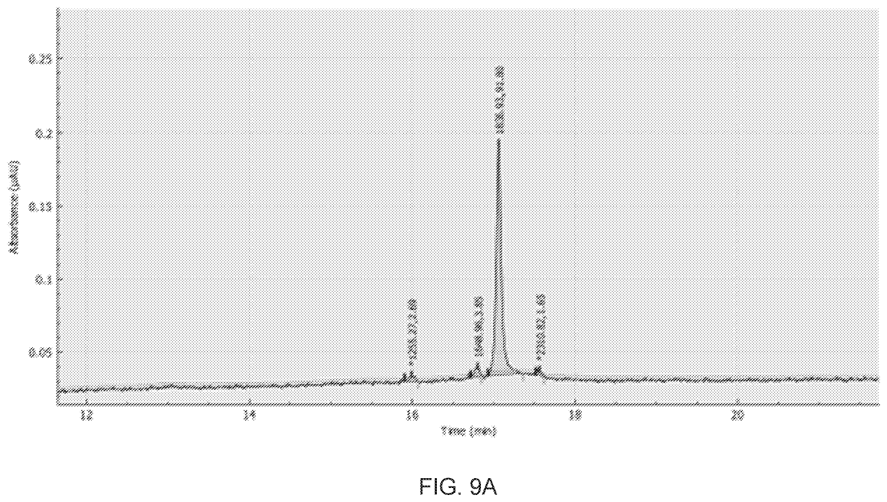

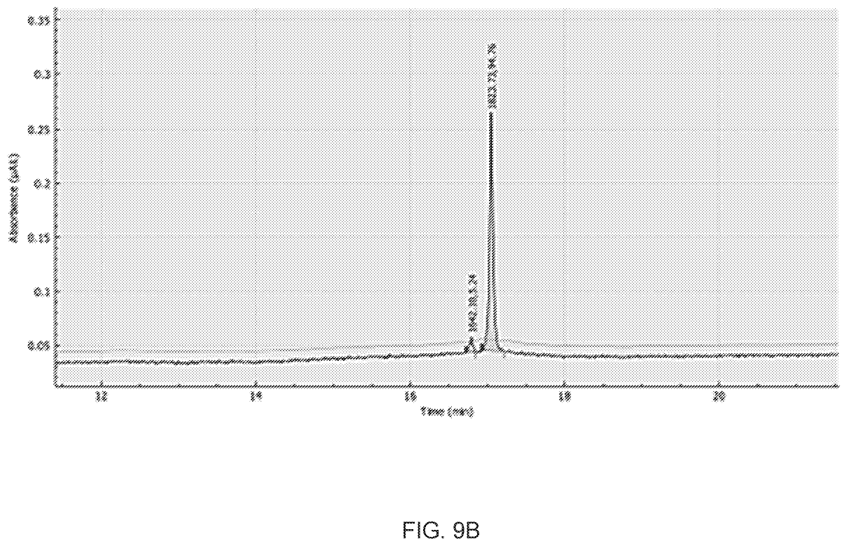

FIGS. 9A-9B are electropherograms displaying the calculated size of LNP-encapsulated mRNA diluted in nuclease-free water without heat treatment (FIG. 9A) or with denaturing at 70° C. (FIG. 9B).

These data demonstrate sample dilution in nuclease-free water and heat denaturing minimize non-specific peaks.

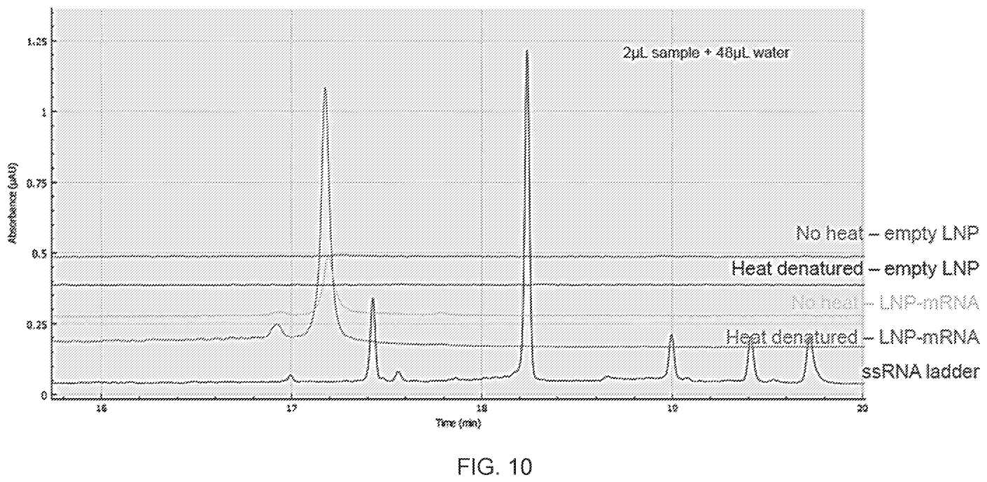

FIG. 10 illustrates an electropherogram of heat-denatured and non-heat denatured LNP-encapsulated mRNA and empty LNPs.

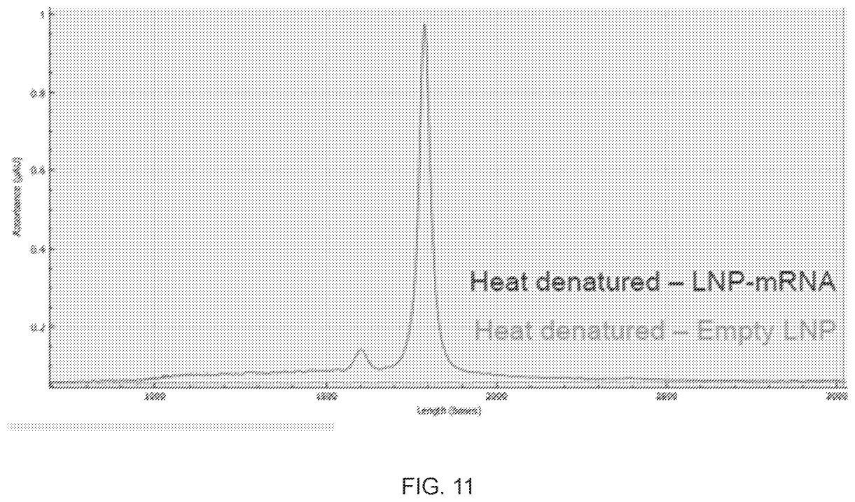

FIG. 11 illustrates an electropherogram of heat-denatured LNP-encapsulated mRNA and an empty LNP diluted in nuclease-free water.

These data demonstrate heat denaturation and sample dilution in nuclease-free water resulted in superior analysis of LNP-encapsulated mRNA. The empty LNP sample (MC3) demonstrates that use of nuclease-free water as a sample diluent did not result in higher molecular weight species, which can interfere with analysis of the mRNA contents.

While the present disclosure has been described with reference to certain embodiments, it will be understood by those skilled in the art that various changes may be made and equivalents may be substituted without departing from the scope of the present disclosure or appended claims. In addition, many modifications may be made to adapt a particular situation or material to the teachings of the present disclosure without departing from its scope. Therefore, it is intended that the present disclosure not be limited to the particular embodiments disclosed, but that the present disclosure will include all aspects falling within the scope of the appended claims.

1. A method for analyzing an encapsulated biomolecule, the method comprising:
loading the encapsulated biomolecule on a capillary electrophoresis (CE) capillary, wherein the CE capillary is filled with a buffer comprising a polymer matrix;
applying a voltage to the CE capillary to cause the encapsulated biomolecule to release a biomolecule from an encapsulating material; and
detecting the biomolecule released from the encapsulating material.

2. The method of claim 1, wherein the polymer matrix comprises a chaotrope.

3. The method of claim 1, wherein detecting the biomolecule released from the encapsulating material produces a set of corresponding values, and the method further comprises quantifying the biomolecule released from the encapsulating material using the corresponding values.

4. The method of claim 1, further comprising adding a fluorescent dye to the polymer matrix and/or to a buffer disposed within the CE capillary, wherein the fluorescent dye binds to the biomolecule resulting in a fluorescently labeled biomolecule.

5. The method of claim 4, wherein the fluorescent dye is a cyanine-based dye.

6. The method of claim 1, further comprising heating the encapsulated biomolecule prior to loading the encapsulated biomolecule on the CE capillary.

7. The method of claim 6, wherein the encapsulated biomolecule is heated at a temperature between about 40° C. to about 90° C., alternatively at a temperature between about 45° C. to about 85° C., alternatively at a temperature between about 50° C. to about 80° C., alternatively at a temperature between about 55° C. to about 78° C., alternatively at a temperature between about 60° C. to about 77° C., alternatively at a temperature between about 65° C. to about 75° C., alternatively at a temperature between about 68° C. to about 74° C., alternatively at a temperature between about 69° C. to about 73° C., alternatively at a temperature of about 70° C.

8. The method of claim 6, wherein the encapsulated biomolecule is heated for at least 2 minutes, alternatively at least 3 minutes, alternatively at least 4 minutes, alternatively at least 5 minutes.

9. The method of claim 6, further comprising cooling the encapsulated biomolecule after heating.

10. The method of claim 1, further comprising treating the encapsulated biomolecule with a denaturing agent prior to loading the encapsulated biomolecule on the CE capillary.

11. The method of claim 1, further comprising detecting the encapsulating material.

12. (canceled)

13. (canceled)

14. (canceled)

15. The method of claim 1, wherein the biomolecule is a protein or a polynucleotide.

16. The method of claim 15, wherein the encapsulating material is a viral vector.

17. The method of claim 1, wherein the polymer matrix is a cross-linked polymer, a linear polymer, a branched polymer, linear polyacrylamide, polyethylene oxide, polyethylene glycol, dextran, or pullulan.

18. The method of claim 1, wherein detecting the biomolecule utilizes a fluorescence detector, optionally wherein the fluorescence detector is a laser-induced fluorescence (LIF) detector, a lamp-based fluorescence detector, or a native fluorescence detector.

19. (canceled)

20. The method of claim 1, wherein the chaotrope is n-butanol, ethanol, guanidinium chloride, lithium acetate, magnesium chloride, 2-propanol, sodium dodecyl sulfate, thiourea, or urea.

21. (canceled)

22. The method of claim 2, wherein the buffer further comprises the chaotrope.

23.-31. (canceled)

32. The method of claim 15, wherein the biomolecule is the polynucleotide.

33. The method of claim 32, wherein the polynucleotide is an mRNA encoding a polypeptide.

34. The method of claim 32, wherein the encapsulating material is a lipid nanoparticle comprising one or more of an ionizable cationic lipid, a PEGylated lipid, a phospholipid, and/or cholesterol.
